(19) United States
(12) Patent Application Publication
(10) Pub. No.: US 2006/0243720 A1
(43) Pub. Date: Nov. 2, 2006

(54) System for keeping a patient warm during the perioperative period

(75) Inventors: Jochim Koch, Ratzeburg (DE); Susanne Stahlkopf, Hamburg (DE)

Correspondence Address: MCGLEW & TUTTLE, PC, P.O. BOX 9227, SCARBOROUGH STATION, SCARBOROUGH, NY, 10510-9227, US

(73) Assignee: Drager Medical AG & Co., KG, Lubeck (DE)

(21) Appl. No.: 11/343,856

(22) Filed: Jan. 31, 2006

(30) Foreign Application Priority Data
Apr. 27, 2005 (DE) 10 2005 019 472.9

(51) Int. Cl. H05B 1/00; A61F 7/00; A61B 5/00

(52) U.S. Cl. 219/217; 600/549; 607/96

(57) ABSTRACT

A system for keeping a patient warm during the perioperative period. The system includes a portable bed (1) for the patient. At least one electric heating pad (3) is provided for keeping the patient on the bed (1) warm. A control device (4), portable with the bed (1), is provided for setting the temperature of the at least one heating pad (3) as a function of the body temperature measured at the patient.

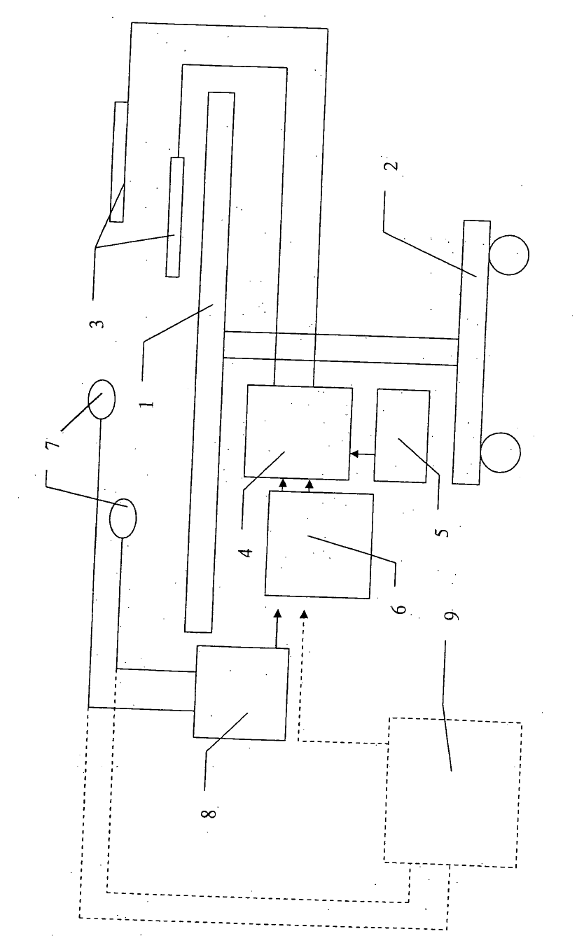

### CROSS REFERENCE TO RELATED APPLICATIONS

This application claims the benefit of priority under 35 U.S.C. § 119 of German Patent Application DE 10 2005 019 472.9 filed Apr. 27, 2005, the entire contents of which are incorporated herein by reference.

### FIELD OF THE INVENTION

The present invention pertains to a system for keeping a patient warm during the perioperative period

### BACKGROUND OF THE INVENTION

It has been known for a long time that patients may cool down relatively greatly before, during and after an operation. Thermotherapy devices, which can make available an additional supply of heat for the patient on the operating table, are therefore available commercially. The heating devices used so far supply heat firstly by convection by supplying warm air via a mattress or blanket, which is permeable to air, to the patient, or secondly, by heat conduction by a mattress or blanket filled with a heat-releasing liquid covering surface areas of the patient, or thirdly, by heat radiation by irradiating the patient with a radiant heater. A certain heating output or a desired temperature is preset and set in the heating devices used so far.

A process for controlling the operating parameters of an incubator is known from DE 198 18 170 C2 (See also corresponding U.S. Pat. No. 6,048,304), wherein the air temperature in the incubator is set as a function of the measured body temperatures of two different skin areas.

DE 100 38 247 C2 pertains to a double temperature sensor for the simultaneous, separate detection of two different temperatures, to which opposite housing parts of the double temperature sensor are exposed.

In contrast to a premature or newborn baby, a patient being operated on does not remain in a room or in an air conditioned area during perioperative hypothermia: For preparation for the operation, the patient is normally pretreated at first in an introduction room. He is placed for this purpose on an operating table in a so-called sluice and is fully undressed. He is then covered with a thin surgical drape only, which has practically no insulating effect, for the surgical procedure proper. The operating rooms are kept, in general, cool, at about 20° C., in order to facilitate the work of the hospital staff. However, this is linked with the consequence that the patient will experience a heat loss and will cool down after a longer time. After the induction of anesthesia, which takes place predominantly in the introduction room, the patient's blood vessels dilate, with the consequence that the colder peripheral blood flows back into the core of the body, whereas the warmer central blood flows into the periphery and somewhat warms same. This effect leads to cooling by one degree Celsius even within the first hour after the induction of the anesthesia. The patient is transported after the surgery into the recovery room, where the patient will finally be placed into a regular bed and will slowly warm up again under an insulating blanket due to the patients own heat.

On the whole, operated-on patients frequently report a very unpleasant and painful trembling caused by cold, which they experienced on waking up from the anesthesia. In addition, wound healing is markedly retarded and the rate of infections is significantly increased in cooled patients, and the total amount of respiration time may even double due to the perioperative hypothermia in artificially respirated patients.

### SUMMARY OF THE INVENTION

Thus, the object of the present invention is to provide a system for keeping a patient warm during the perioperative period.

According to the invention, a system for keeping a patient warm during the perioperative period is provided. The system comprises a portable bed for the patient with an associated electric heating pad for keeping the patient warm on the bed. A control device is provided that is portable with the bed for setting the temperature of the heating pad as a function of the patient body temperature measured at the patient.

The control device may advantageously be equipped with an interface for a temperature-measuring and display device that can be connected in a modular manner. The temperature-measuring and display device may advantageously receive measured signals of a temperature sensor located at the patient's body. The temperature-measuring and display device may advantageously receive measured signals of plural sensors such as a first temperature sensor, a central temperature sensor and a second, peripheral temperature sensor located at the patient's body. The temperature sensor may advantageously comprise a double temperature sensor.

The control device may advantageously be equipped with an interface for a patient monitor for detecting the patient's vital parameters. The interface may also be connected in a modular manner. The interface may be a wireless, wired or another interface. Advantageously the interface is infrared. WiFi (IEEE 802.11 etc.) and similar wireless formats may be used.

The heating pad may be a mattress with an electric heater. A cover is advantageously used in combination to cover the patient, with the cover including a second heating pad.

The electric heating pad is advantageously used to keep the patient warm as a function of the patient's body temperature measured by means the temperature sensor and is set by means of the control device after a set point-actual value comparison.

The portable bed is advantageously a movable operating table.

An essential advantage of the system for keeping a patient warm during the perioperative period is that the patient is kept warm continuously in a heating device from the introduction to the recovery room via the operating room and the patient's body temperature can be both monitored and controlled. Contrary to heating outputs or desired temperatures set at a fixed value, the system can respond to the individual heat losses of the patient and counteract a possible drop in temperature. The system has a modular design for this purpose, so that, on the one hand, it can be transported through the different rooms together with the patient on the portable bed, especially an operating table, and can be connected to different existing monitor systems, especially also in a wireless manner via a corresponding interface. The functions remain inherently safe, even if an interface connection is interrupted.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

The only FIGURE is a schematic view of a system according to the invention.

### DESCRIPTION OF THE PREFERRED EMBODIMENTS

Referring to the drawings in particular, The portable bed 1 is intended for accommodating a patient. The bed 1 is an operating table movable by means of a chassis 2 with wheels in the example. The patient is kept warm by means of electrically heatable heating pads, and a heating pad 3 may be a mattress with an electric heater and a second heating pad 3 is used to cover the patient. The control device 4 for the heating pads 3 is docked at the operating table and can be transported together with the patient and the bed 1. On the way between the rooms, the control device 4 and the heating pads 3 can be optionally supplied with electricity by means of batteries 5. The control device 4 may be set at a set point for the mattress heating. The control device 4 has a display for displaying the values as well as for the output of an alarm.

A connectable module for the body temperature measurement is provided for the body temperature control with the heating pads 3. This unit is provided separately for the control device 4, so that the display with set points, instantaneous values and optionally alarms can be better viewed and operated by the anesthesiologist, for example, at the head end of the bed 1, or the operating table. For example, two temperature sensors 7 are used to determine the patient's peripheral and central body temperatures. The measured signals enter the modular temperature-measuring and display device 8. The set points for the patient's two temperatures can be entered in the temperature-measuring and display device 8. The heater in the heating pads 3 can be actuated directly externally via the interface 6. The interface 6 is especially a wireless infrared interface. The heater in the heating pads 3 is actuated externally by a set point-actual value comparison and the temperature is adjusted to the set point. To establish an inherently safe body temperature control, the control device 4 is designed such that it always uses its set internal set point of the pad and/or mattress heater temperature as a reference when the interface 6 is not occupied or is interrupted. Thus, the heater operates autonomously and avoids overheating the patient. As an alternative, the interface 6 may also be occupied by a patient monitor 9 for the patient's vital parameters, which can be connected in a modular manner. As another alternative, the temperature sensors 7 are connected to such a patient monitor 9. The set points for the body temperature are likewise entered via the patient monitor 9 in this case. A safety concept, which adjusts the control 4 to the internal maximum temperature of the heating pads 3 or the mattress in case of interruption of the function or when the patient monitor 9 is not connected, is provided here as well.

While specific embodiments of the invention have been shown and described in detail to illustrate the application of the principles of the invention, it will be understood that the invention may be embodied otherwise without departing from such principles.

What is claimed is:

1. A system for keeping a patient warm during the perioperative period, the system comprising:
a portable bed for the patient;
an electric heating pad for keeping the patient warm on said bed; and
a control device portable with said bed for setting the temperature of said heating pad as a function of the patient body temperature measured at the patient.

2. A system in accordance with claim 1, wherein said control device is equipped with an interface for a temperature-measuring and display device that can be connected in a modular manner.

3. A system in accordance with claim 1, wherein said control device is equipped with an interface for a patient monitor for detecting the patient's vital parameters, which can be connected in a modular manner.

4. A system in accordance with claim 2, wherein said interface is an infrared interface.

5. A system in accordance with claim 2, wherein said temperature-measuring and display device receives measured signals of a temperature sensor located at the patient's body.

6. A system in accordance with claim 5, wherein said temperature-measuring and display device receives measured signals of a first temperature sensor, a central temperature sensor and a second, peripheral temperature sensor located at the patient's body.

7. A system in accordance with claim 5, wherein said temperature sensor comprises a double temperature sensor.

8. A system in accordance with claim 1, wherein said heating pad is a mattress with an electric heater and further comprising a cover used to cover the patient, said cover including a second heating pad.

9. A system in accordance with claim 5, wherein said electric heating pad is used to keep the patient warm as a function of the patient's body temperature measured by means said temperature sensor and is set by means of said control device after a set point-actual value comparison.

10. A system in accordance with claim 1, wherein said portable bed is a movable operating table.

11. A system for keeping a patient warm the system comprising:
a patient bed unit comprising a movable chassis with wheels, the chassis supporting a bed having a mattress with a bed surface for the patient to lie thereon, the mattress having an electric heater;
a temperature-measuring device including a temperature sensor for measuring temperature at the patient to provide a body temperature measurement; and
a control device operatively connected to the patient bed unit and being positionable for movement therewith, said control device regulating power to said electric heater for setting the temperature of said electric heater as a function of said body temperature measurement.

12. A system in accordance with claim 11, wherein said control device is connected with an interface, said temperature-measuring device being connected to said interface in a modular manner.

13. A system in accordance with claim 12, wherein said temperature-measuring device is associated with a display device for displaying said body temperature measurement.

14. A system in accordance with claim 11, further comprising a patient monitor system for detecting vital parameters of the patient, wherein said control device is connected with an interface, said patient monitor system being connected to said interface in a modular manner.

15. A system in accordance with claim 14, wherein said interface is an infrared interface.

16. A system in accordance with claim 11, wherein said temperature sensor comprises a set of patient body location sensors including a peripheral patient body portion temperature sensor and a central patient body portion temperature sensor.

17. A system in accordance with claim 11, wherein said temperature sensor comprises a double temperature sensor for providing a first body temperature measurement and a second temperature measurement.

18. A system in accordance with claim 11, further comprising a cover associated with said patient bed unit and movable with said patient bed unit, said cover being used to cover said patient, said cover including a cover electric heater operatively connected to said control device.

19. A system in accordance with claim 12, wherein at least one of said temperature-measuring device and another device connected to said interface includes means for setting a set point of said electric heater and said control device compares one or more temperatures measured by said temperature measuring device to said set point for a set point-actual value comparison for regulating power to said electric heater.

20. A system in accordance with claim 11, wherein said patient bed unit comprises an operating table.
